(19) United States
(12) Patent Application Publication
(10) Pub. No.: US 2018/0147148 A1
(43) Pub. Date: May 31, 2018

(54) PHARMACEUTICAL COMPOSITION OF TERIFLUNOMIDE

(71) Applicant: FTF PHARMA PRIVATE LIMITED, Sanand dist.: Gujarat, Ahmedabad (IN)

(72) Inventors: Tarun Kumar SATPATHY, Talsara Dist-Sundargarh State Talsara (IN); Dhiren J. PATEL, Modasa Dist- Arravalli (Modasa) State (IN); Jayanta Kumar MANDAL, Ramdev Nagar, Satelite, Ahmedabad (IN)

(73) Assignee: FTF PHARMA PRIVATE LIMITED, Sanand dist.: Gujarat, Ahmedabad (IN)

(21) Appl. No.: 15/575,350

(22) PCT Filed: May 11, 2016
(86) PCT No.: PCT/IB2016/052693

§ 371 Date: Nov. 18, 2017

(30) Foreign Application Priority Data
May 23, 2015 (IN) 2013/MUM/2015

(51) Int. Cl. A61K 9/20; A61K 31/277

(52) U.S. Cl. CPC A61K 9/2013; A61K 31/277; A61K 9/2009; A61K 9/2054; A61K 9/2059; A61K 9/2018

(57) ABSTRACT

The invention relates to the formulation of solid pharmaceutical compounds. Further, solid pharmaceutical composition comprising a therapeutically effective amount of teriflunomide or a pharmaceutically acceptable basic addition salt thereof and excipients.

### FIELD OF THE INVENTION

Present invention relates to a pharmaceutically acceptable oral dosage form comprising a therapeutically effective amount of teriflunomide or a pharmaceutically acceptable salt thereof and pharmaceutically acceptable excipients. Further, stability of composition is maintained without use of basic substance and pH of composition is more than 3.5

### BACKGROUND OF THE INVENTION

Teriflunomide is 2-cyano-3-hydroxy-n-[4-(trifluoromethyl)phenyl]-2-butenamide having chemical formula is C₁₂H₉F₃N₂O₂, molecular weight is 270.207 and chemical structure illustrated in Formula I:

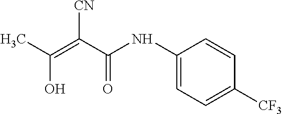 [SMILES: C/C(O)=C(\C#N)C(=O)Nc1ccc(C(F)(F)F)cc1]

Teriflunomide is an immunomodulatory drug inhibiting pyrimidine de novo synthesis by blocking the enzyme dihydroorotate dehydrogenase. Further, Teriflunomide inhibits rapidly dividing cells, including activated T cells. Teriflunomide is useful for treatment of autoimmune diseases in particular systemic lupus erythematosus or chronic graft-versus-host disease, multiple sclerosis or rheumatoid arthritis.

EP1381356 discloses the use of Teriflunomide for the manufacture of a medicament for treating multiple sclerosis wherein said medicament is administered orally.

WO2013062442 discloses composition containing Teriflunomide with colloidal silicon dioxide which is present between the range of 0.8-1.2% w/w. Said compositions show a slighter degradation of teriflunomide.

U.S. Pat. No. 8,802,735 discloses Teriflunomide containing solid pharmaceutical composition without colloidal silicon dioxide. Further, pH of said pharmaceutical composition is not more than 2.2.

### OBJECT OF THE INVENTION

Primary object of the present invention is to provide pharmaceutically acceptable oral dosage form comprising a therapeutically effective amount of teriflunomide or a pharmaceutically acceptable salt thereof and pharmaceutically acceptable excipients with use of aerosil and without use of basic ingredients.

Second object of the present invention is to provide process for preparation of pharmaceutically acceptable oral dosage form comprising a therapeutically effective amount of teriflunomide or a pharmaceutically acceptable salt thereof and pharmaceutically acceptable excipients.

### DETAIL DESCRIPTION OF THE INVENTION

The present invention relates to solid pharmaceutically acceptable dosage form composition comprising an active principle teriflunomide or a pharmaceutically acceptable salt and other excepients. Further, teriflunomide or a pharmaceutically acceptable salt are present within the range of 7 to 11% w/w and excepients can be selected from disintigrant, diluents and lubricant or glidant etc.

In further aspect of present invention diluents selected from the group of cellulose, cellulose acetate, dextrates, dextrin, dextrose, fructose, 1-O-α-D-Glucopyranosyl-D-mannitol, glyceryl palmitostearate, hydrogenated vegetable oil, kaolin, lactitol, lactose, lactose mono-hydrate, maltitol, mannitol, maltodextrin, maltose, pregelatinized starch, sodium chloride, sorbitol, starches, sucrose, talc and xylitol or a mixture of one or more of said diluents.

Disintegrants used for the preparation of solid oral dosage form are selected from the group of carboxymethylcellulose, low substituted hydroxypropyl cellulose, microcrystalline cellulose, powdered cellulose, croscarmellose sodium, methylcellulose, polacrilin potassium, sodium alginate, and sodium starch glycolate or a mixture of one or more of said disintegrants.

Lubricants selected from the group of calcium stearate, glyceryl palmitostearate, sodium benzoate, sodium lauryl sulfate, sodium stearyl fumarate, stearic acid, talc, zinc stearate and magnesium stearate or a mixture of one or more of said lubricants.

In other embodiment solid pharmaceutical composition further comprising about 0% to 0.5% w/w colloidal silicon dioxide (aerosil).

Further, the invention relate to a solid pharmaceutical composition comprising a therapeutically effective amount of teriflunomide or pharmaceutically acceptable basic addition salt thereof, wherein the pH of the solid pharmaceutical composition is more than 3.5.

The pH determination is performed by suspending solid dosage form in about 1 ml of purified water. The pH of supernatant is determined with a pH sensitive probe.

The present invention has been described by way of examples only and it is recognized that modifications are falling within the scope and spirit of the appended claims, and which would be obvious for a person skilled in the art based upon the disclosure herein, are also considered to be included within the scope of this invention. The invention is exemplified via its two batched showing with or without use of colloidal silicon dioxide.

Drug or excepients with colloidal silicon dioxide with its range are shown below in table:

| Sr. No. | Drug/Excepients | % w/w |
|---|---|---|
| 1 | Teriflunomide | 7 to 11 |
| 2 | Lactose monohydrate | 20 to 60 |
| 3 | Microcrystalline cellulose | 5 to 50 |
| 4 | Sodium starch glycolate | 1 to 6 |
| 5 | Hydroxypropylcellulose | 1 to 6 |
| 6 | Starch | 0 to 8 |
| 7 | Colloidal silicon dioxide | 0 to 0.5 |
| 8 | Magnesium stearate | 0.25 to 1.0 |
| | Total | 100 |

The composition can be compressed in tablet or can be filled in capsule.

| | | Batch No. | | | |
|---|---|---|---|---|---|
| | | TERT2002 | | TERT2003 | |
| | | Rationale | | | |
| | | Without | | | |
| | | Colloidal | | | |
| | | Silicon | | With Colloidal | |
| | | Dioxide | | Silicon Dioxide | |
| Sr. No. | Ingredients | mg/unit | % w/w | mg/unit | % w/w |
| 1 | Teriflunomide | 14.000 | 9.333 | 14.000 | 9.333 |
| 2 | Lactose monohydrate | 72.000 | 48.000 | 72.000 | 48.000 |
| 3 | Microcrystalline | 48.000 | 32.000 | 53.850 | 35.900 |
| | cellulose | | | | |
| 4 | Sodium starch glycolate | 6.000 | 4.000 | 6.000 | 4.000 |
| 5 | Hydroxypropylcellulose | 3.000 | 2.000 | 3.000 | 2.000 |
| 6 | Starch | 6.000 | 4.000 | 0.000 | 0.000 |
| 7 | Colloidal Silicon Dioxide | 0.00 | 0.00 | 0.150 | 0.100 |
| 8 | Magnesium stearate | 1.000 | 0.667 | 1.000 | 0.667 |
| | Total weight of Tablet | 150.000 | 100.000 | 150.000 | 100.000 |

Tablet is prepared by conventional method and analyzed for impurity profiles.

Impurity profiles results are shown as below:

| | Related | | | |
|---|---|---|---|---|
| Batch No. | Substances | Initial | 3 M 40/75 | 3 M 25/60 |
| TERT2002 | Impurity A | ND | ND | ND |
| | Impurity B | ND | ND | ND |
| | Impurity C | ND | ND | ND |
| | Single Max Imp | 0.05 | 0.17 | 0.04 |
| | Total Impurity | 0.07 | 0.21 | 0.06 |
| TERT2003 | Impurity A | ND | ND | ND |
| | Impurity B | ND | ND | ND |
| | Impurity C | ND | ND | ND |
| | Single Max Imp | 0.05 | 0.16 | 0.05 |
| | Total Impurity | 0.07 | 0.21 | 0.08 |

We claim:

1. A pharmaceutical composition comprising of teriflunomide or pharmaceutically acceptable salt thereof and pharmaceutically acceptable excepients having pH more than 3.5 without use of basic ingredient.

2. A pharmaceutical composition as claimed in claim 1, wherein the composition comprises of a) 7% to 11% w/w teriflunomide, or a pharmaceutically acceptable basic addition salt thereof, b) 1% to 50% w/w disintegrant, c) 0% to 40% w/w binder, d) 0.1% to 2% w/w lubricant, and e) diluents; f) colloidal silicon dioxide between 0 to 0.5%.

3. A solid pharmaceutical composition as claimed in claim 1, wherein disintegrant can be one or more selected from the group of carboxymethylcellulose, low substituted hydroxypropyl cellulose, microcrystalline cellulose, powdered cellulose, crosscarmellose sodium, methylcellulose, polacrilin potassium, sodium alginate, and sodium starch glycolate

4. A solid pharmaceutical composition as claimed in claim 1, wherein diluents can be one or more selected from the group of cellulose, cellulose acetate, dextrates, dextrin, dextrose, fructose, 1-O-α-D-Glucopyranosyl-D-mannitol, glyceryl palmitostearate, hydrogenated vegetable oil, kaolin, lactitol, lactose, lactose mono-hydrate, maltitol, mannitol, maltodextrin, maltose, pregelatinized starch, sodium chloride, sorbitol, starches, sucrose, talc and xylitol.

5. A pharmaceutical composition as claimed in claim 1, wherein lubricant can be one or more selected from the group of calcium stearate, glyceryl palmitostearate, sodium benzoate, sodium lauryl sulfate, sodium stearyl fumarate, stearic acid, talc, zinc stearate and magnesium stearate.

6. A pharmaceutical composition as claimed in claim 1, wherein the composition can be compressed in tablet or can be filled in capsule.

7. A pharmaceutical composition as claimed in claims 1 and 6, wherein tablet is prepared by conventional method.
